SEQUENCE LISTING (208 sequences)

SEQ ID NO: 1 - RNA, 29 nt, Artificial sequence
  Adaptor sequence
  misc_feature (26)..(29): n is a, c, g, or u
gaacgacauggcuacgauccgacuunnnn

SEQ ID NO: 2 - DNA, 45 bp, Artificial sequence
  Reverse transcription primer sequence
  misc_feature (45)..(45): n is a, c, g, or t
gaccgcttggcctccgactttttttttttttttttttttttttvn

SEQ ID NO: 3 - DNA, 59 bp, Artificial sequence
  Primer sequence
  misc_feature (18)..(27): n is a, c, g, or t
tgtgagccaaggagttgnnnnnnnnnnttgtcttcctaagaccgcttggcctccgactt

SEQ ID NO: 4 - DNA, 25 bp, Artificial sequence
  Primer sequence
gaacgacatggctacgatccgactt

SEQ ID NO: 5 - DNA, 50 bp, Artificial sequence
  Probe sequence
taatgatccttccgcaggttcacctacggaaaccttgttacgacttttac

SEQ ID NO: 6 - DNA, 50 bp, Artificial sequence
  Probe sequence
ttcctctagatagtcaagttcgaccgtcttctcagcgctccgccagggcc

SEQ ID NO: 7 - DNA, 50 bp, Artificial sequence
  Probe sequence
gtgggccgaccccggcggggccgatccgagggcctcactaaaccatccaa

SEQ ID NO: 8 - DNA, 50 bp, Artificial sequence
  Probe sequence
tcggtagtagcgacgggcggtgtgtacaaagggcagggacttaatcaacg

SEQ ID NO: 9 - DNA, 50 bp, Artificial sequence
  Probe sequence
caagcttatgacccgcacttactcgggaattccctcgttcatggggaata

SEQ ID NO: 10 - DNA, 50 bp, Artificial sequence
  Probe sequence
attgcaatccccgatccccatcacgaatggggttcaacgggttacccgcg

SEQ ID NO: 11 - DNA, 50 bp, Artificial sequence
  Probe sequence
cctgccggcgtagggtaggcacacgctgagccagtcagtgtagcgcgcgt

SEQ ID NO: 12 - DNA, 50 bp, Artificial sequence
  Probe sequence
gcagccccggacatctaagggcatcacagacctgttattgctcaatctcg

SEQ ID NO: 13 - DNA, 50 bp, Artificial sequence
  Probe sequence
ggtggctgaacgccacttgtccctctaagaagttgggggacgccgaccgc

SEQ ID NO: 14 - DNA, 50 bp, Artificial sequence
  Probe sequence
tcgggggtcgcgtaactagttagcatgccagagtctcgttcgttatcgga

SEQ ID NO: 15 - DNA, 50 bp, Artificial sequence
  Probe sequence
attaaccagacaaatcgctccaccaactaagaacggccatgcaccaccac

SEQ ID NO: 16 - DNA, 50 bp, Artificial sequence
  Probe sequence
ccacggaatcgagaaagagctatcaatctgtcaatcctgtccgtgtccgg

SEQ ID NO: 17 - DNA, 50 bp, Artificial sequence
  Probe sequence
gccgggtgaggtttcccgtgttgagtcaaattaagccgcaggctccactc

SEQ ID NO: 18 - DNA, 50 bp, Artificial sequence
  Probe sequence
ctggtggtgcccttccgtcaattcctttaagtttcagctttgcaaccata

SEQ ID NO: 19 - DNA, 50 bp, Artificial sequence
  Probe sequence
ctccccccggaacccaaagactttggtttcccggaagctgcccggcgggt

SEQ ID NO: 20 - DNA, 50 bp, Artificial sequence
  Probe sequence
catgggaataacgccgccgcatcgccggtcggcatcgtttatggtcggaa

SEQ ID NO: 21 - DNA, 50 bp, Artificial sequence
  Probe sequence
ctacgacggtatctgatcgtcttcgaacctccgactttcgttcttgatta

SEQ ID NO: 22 - DNA, 50 bp, Artificial sequence
  Probe sequence
atgaaaacattcttggcaaatgctttcgctctggtccgtcttgcgccggt

SEQ ID NO: 23 - DNA, 50 bp, Artificial sequence
  Probe sequence
ccaagaatttcacctctagcggcgcaatacgaatgcccccggccgtccct

SEQ ID NO: 24 - DNA, 50 bp, Artificial sequence
  Probe sequence
cttaatcatggcctcagttccgaaaaccaacaaaatagaaccgcggtcct

SEQ ID NO: 25 - DNA, 50 bp, Artificial sequence
  Probe sequence
attccattattcctagctgcggtatccaggcggctcgggcctgctttgaa

SEQ ID NO: 26 - DNA, 50 bp, Artificial sequence
  Probe sequence
cactctaattttttcaaagtaaacgcttcgggccccgcgggacactcagc

SEQ ID NO: 27 - DNA, 50 bp, Artificial sequence
  Probe sequence
taagagcatcgagggggcgccgagaggcaaggggcggggacgggcggtgg

SEQ ID NO: 28 - DNA, 50 bp, Artificial sequence
  Probe sequence
ctcgcctcgcggcggaccgcccgcccgctcccaagatccaactacgagct

SEQ ID NO: 29 - DNA, 50 bp, Artificial sequence
  Probe sequence
ttttaactgcagcaactttaatatacgctattggagctggaattaccgcg

SEQ ID NO: 30 - DNA, 50 bp, Artificial sequence
  Probe sequence
gctgctggcaccagacttgccctccaatggatcctcgttaaaggatttaa

SEQ ID NO: 31 - DNA, 50 bp, Artificial sequence
  Probe sequence
agtggactcattccaattacagggcctcgaaagagtcctgtattgttatt

SEQ ID NO: 32 - DNA, 50 bp, Artificial sequence
  Probe sequence
tttcgtcactacctccccgggtcgggagtgggtaatttgcgcgcctgctg

SEQ ID NO: 33 - DNA, 50 bp, Artificial sequence
  Probe sequence
ccttccttggatgtggtagccgtttctcaggctccctctccggaatcgaa

SEQ ID NO: 34 - DNA, 50 bp, Artificial sequence
  Probe sequence
ccctgattccccgtcacccgtggtcaccatggtaggcacggcgactacca

SEQ ID NO: 35 - DNA, 48 bp, Artificial sequence
  Probe sequence
tcgaaagttgatagggcagacgttcgaatgggtcgtcgccgccacggg

SEQ ID NO: 36 - DNA, 50 bp, Artificial sequence
  Probe sequence
gcgtgcgatcggcccgaggttatctagagtcaccaaagccgccggcgccc

SEQ ID NO: 37 - DNA, 50 bp, Artificial sequence
  Probe sequence
gccccccggccggggccggagaggggctgaccgggttggttttgatctga

SEQ ID NO: 38 - DNA, 50 bp, Artificial sequence
  Probe sequence
taaatgcacgcatcccccccgcgaagggggtcagcgcccgtcggcatgta

SEQ ID NO: 39 - DNA, 50 bp, Artificial sequence
  Probe sequence
ttagctctagaattaccacagttatccaagtaggagaggagcgagcgacc

SEQ ID NO: 40 - DNA, 50 bp, Artificial sequence
  Probe sequence
aaaggaaccataactgatttaatgagccattcgcagtttcactgtaccgg

SEQ ID NO: 41 - DNA, 50 bp, Artificial sequence
  Probe sequence
ccgtgcgtacttagacatgcatggcttaatctttgagacaagcatatgct

SEQ ID NO: 42 - DNA, 50 bp, Artificial sequence
  Probe sequence
tggcttaatctttgagacaagcatatgctactggcaggatcaaccaggta

SEQ ID NO: 43 - DNA, 50 bp, Artificial sequence
  Probe sequence
gacaaacccttgtgtcgagggctgactttcaatagatcgcagcgagggag

SEQ ID NO: 44 - DNA, 50 bp, Artificial sequence
  Probe sequence
ctgctctgctacgtacgaaaccccgacccagaagcaggtcgtctacgaat

SEQ ID NO: 45 - DNA, 49 bp, Artificial sequence
  Probe sequence
ggtttagcgccaggttccccacgaacgtgcggtgcgtgacgggcgaggg

SEQ ID NO: 46 - DNA, 50 bp, Artificial sequence
  Probe sequence
gcggccgcctttccggccgcgccccgtttcccaggacgaagggcactccg

SEQ ID NO: 47 - DNA, 50 bp, Artificial sequence
  Probe sequence
caccggaccccggtcccggcgcgcggcggggcacgcgccctcccgcggcg

SEQ ID NO: 48 - DNA, 50 bp, Artificial sequence
  Probe sequence
gggcgcgtggaggggrgggcggcccgccggcggggacaggcgggggaccg

SEQ ID NO: 49 - DNA, 50 bp, Artificial sequence
  Probe sequence
gctatccgaggccaaccgaggctccgcggcgctgccgtatcgttcgcctg

SEQ ID NO: 50 - DNA, 50 bp, Artificial sequence
  Probe sequence
ggcgggattctgacttagaggcgttcagtcataatcccacagatggtagc

SEQ ID NO: 51 - DNA, 50 bp, Artificial sequence
  Probe sequence
ttcgccccattggctcctcagccaagcacatacaccaaatgtctgaacct

SEQ ID NO: 52 - DNA, 50 bp, Artificial sequence
  Probe sequence
gcggttcctctcgtactgagcaggattaccatggcaacaacacatcatca

SEQ ID NO: 53 - DNA, 50 bp, Artificial sequence
  Probe sequence
gtagggtaaaactaacctgtctcacgacggtctaaacccagctcacgttc

SEQ ID NO: 54 - DNA, 50 bp, Artificial sequence
  Probe sequence
cctattagtgggtgaacaatccaacgcttggcgaattctgcttcacaatg

SEQ ID NO: 55 - DNA, 50 bp, Artificial sequence
  Probe sequence
ataggaagagccgacatcgaaggatcaaaaagcgacgtcgctatgaacgc

SEQ ID NO: 56 - DNA, 50 bp, Artificial sequence
  Probe sequence
ttggccgccacaagccagttatccctgtggtaacttttctgacacctcct

SEQ ID NO: 57 - DNA, 50 bp, Artificial sequence
  Probe sequence
gcttaaaacccaaaaggtcagaaggatcgtgaggccccgctttcacggtc

SEQ ID NO: 58 - DNA, 50 bp, Artificial sequence
  Probe sequence
tgtattcgtactgaaaatcaagatcaagcgagcttttgcccttctgctcc

SEQ ID NO: 59 - DNA, 50 bp, Artificial sequence
  Probe sequence
acgggaggtttctgtcctccctgagctcgccttaggacacctgcgttacc

SEQ ID NO: 60 - DNA, 50 bp, Artificial sequence
  Probe sequence
gtttgacaggtgtaccgccccagtcaaactccccacctggcactgtcccc

SEQ ID NO: 61 - DNA, 50 bp, Artificial sequence
  Probe sequence
ggagcgggtcgcgcccggccgggcgggcgcttggcgccagaagcgagagc

SEQ ID NO: 62 - DNA, 50 bp, Artificial sequence
  Probe sequence
ccctcgggctcgcccccccgcctcaccgggtcagtgaaaaaacgatcaga

SEQ ID NO: 63 - DNA, 50 bp, Artificial sequence
  Probe sequence
gtagtggtatttcaccggcggcccgcagggccgcggaccccgccccgggc

SEQ ID NO: 64 - DNA, 50 bp, Artificial sequence
  Probe sequence
ccctcgcggggacaccgggrgggcgccgggggcctcccacttattctaca

SEQ ID NO: 65 - DNA, 50 bp, Artificial sequence
  Probe sequence
cctctcatgtctcttcaccgtgccagactagagtcaagctcaacagggtc

SEQ ID NO: 66 - DNA, 50 bp, Artificial sequence
  Probe sequence
ttctttccccgctgattccgccaagcccgttcccttggctgtggtttcgc

SEQ ID NO: 67 - DNA, 50 bp, Artificial sequence
  Probe sequence
tggatagtaggtagggacagtgggaatctcgttcatccattcatgcgcgt

SEQ ID NO: 68 - DNA, 50 bp, Artificial sequence
  Probe sequence
cactaattagatgacgaggcatttggctaccttaagagagtcatagttac

SEQ ID NO: 69 - DNA, 50 bp, Artificial sequence
  Probe sequence
tcccgccgtttacccgcgcttcattgaatttcttcactttgacattcaga

SEQ ID NO: 70 - DNA, 50 bp, Artificial sequence
  Probe sequence
gcactgggcagaaatcacatcgcgtcaacacccgccgcgggccttcgcga

SEQ ID NO: 71 - DNA, 50 bp, Artificial sequence
  Probe sequence
tgctttgttttaattaaacagtcggattcccctggtccgcaccagttcta

SEQ ID NO: 72 - DNA, 50 bp, Artificial sequence
  Probe sequence
agtcggctgctaggcgccggccgaggcgaggcgcgcgcggaaccgcggcc

SEQ ID NO: 73 - DNA, 50 bp, Artificial sequence
  Probe sequence
ccgggggcggacccggcgggrgggaccggcccgcggcccctccgccgcct

SEQ ID NO: 74 - DNA, 50 bp, Artificial sequence
  Probe sequence
gccgccgccgccgccgcgcgccgaggaggaggggggaacggggggcggac

SEQ ID NO: 75 - DNA, 49 bp, Artificial sequence
  Probe sequence
gggccgggrgggtagggcggggggacgaaccgccccgccccgccgcccg

SEQ ID NO: 76 - DNA, 50 bp, Artificial sequence
  Probe sequence
ccgaccgccgccgcccgaccgctcccgcccccagcggacgcgcgcgcgac

SEQ ID NO: 77 - DNA, 50 bp, Artificial sequence
  Probe sequence
cgagacgtggggtgggggtggggggcgcgccgcgccgccgccgggctccc

SEQ ID NO: 78 - DNA, 50 bp, Artificial sequence
  Probe sequence
cgggggcggccgcgacgcccgccgcagctggggcgatccacgggaagggc

SEQ ID NO: 79 - DNA, 50 bp, Artificial sequence
  Probe sequence
ccggctcgcgtccagagtccgcgccgccgccggccccccgggtccccggg

SEQ ID NO: 80 - DNA, 50 bp, Artificial sequence
  Probe sequence
gcccccctcgcggggacctgcccccgccggccgccccggcggccgccgcg

SEQ ID NO: 81 - DNA, 50 bp, Artificial sequence
  Probe sequence
cggcccctgccgccccgacccttctccccccgccgcgcccccacgcggcg

SEQ ID NO: 82 - DNA, 50 bp, Artificial sequence
  Probe sequence
ctcccccggggaggggggaggacggggagcgggggagagagagagagaga

SEQ ID NO: 83 - DNA, 50 bp, Artificial sequence
  Probe sequence
gggcgcggggtggggagggagcgagcggcgcgcgcgggtggggcggggga

SEQ ID NO: 84 - DNA, 50 bp, Artificial sequence
  Probe sequence
gggccgcgaggggggtgccccgggcgtggggrgggcgcgcgcctcgtcca

SEQ ID NO: 85 - DNA, 50 bp, Artificial sequence
  Probe sequence
gccgcggcgcgcgcccagccccgcttcgcgccccagcccgaccgacccag

SEQ ID NO: 86 - DNA, 50 bp, Artificial sequence
  Probe sequence
cccttagagccaatccttatcccgaagttacggatccggcttgccgactt

SEQ ID NO: 87 - DNA, 50 bp, Artificial sequence
  Probe sequence
cccttacctacattgttccaacatgccagaggctgttcaccttggagacc

SEQ ID NO: 88 - DNA, 50 bp, Artificial sequence
  Probe sequence
tgctgcggatatgggtacggcccggcgcgagatttacaccctctcccccg

SEQ ID NO: 89 - DNA, 50 bp, Artificial sequence
  Probe sequence
gattttcaagggccagcgagagctcaccggacgccgccggaaccgcgacg

SEQ ID NO: 90 - DNA, 50 bp, Artificial sequence
  Probe sequence
ctttccaaggcacgggcccctctctcggggcgaacccattccagggcgcc

SEQ ID NO: 91 - DNA, 50 bp, Artificial sequence
  Probe sequence
ctgcccttcacaaagaaaagagaactctccccggggctcccgccggcttc

SEQ ID NO: 92 - DNA, 50 bp, Artificial sequence
  Probe sequence
tccgggatcggtcgcgttaccgcactggacgcctcgcggcgcccatctcc

SEQ ID NO: 93 - DNA, 50 bp, Artificial sequence
  Probe sequence
gccactccggattcggggatctgaacccgactccctttcgatcggccgag

SEQ ID NO: 94 - DNA, 50 bp, Artificial sequence
  Probe sequence
ggcaacggaggccatcgcccgtcccttcggaacggcgctcgcccatctct

SEQ ID NO: 95 - DNA, 50 bp, Artificial sequence
  Probe sequence
caggaccgactgacccatgttcaactgctgttcacatggaacccttctcc

SEQ ID NO: 96 - DNA, 50 bp, Artificial sequence
  Probe sequence
acttcggccttcaaagttctcgtttgaatatttgctactaccaccaagat

SEQ ID NO: 97 - DNA, 50 bp, Artificial sequence
  Probe sequence
ctgcacctgcggcggctccacccgggcccgcgccctaggcttcaaggctc

SEQ ID NO: 98 - DNA, 50 bp, Artificial sequence
  Probe sequence
accgcagcggccctcctactcgtcgcggcgtagcgtccgcggggctccgg

SEQ ID NO: 99 - DNA, 45 bp, Artificial sequence
  Probe sequence
gggcggggagcggggcgtgggcgggaggaggggaggaggcgtggg

SEQ ID NO: 100 - DNA, 50 bp, Artificial sequence
  Probe sequence
gggcgggggaaggaccccacacccccgccgccgccgccgccgccgccctc

SEQ ID NO: 101 - DNA, 50 bp, Artificial sequence
  Probe sequence
cgacgcacaccacacgcgcgcgcgcgcgcgccgcccccgccgctcccgtc

SEQ ID NO: 102 - DNA, 50 bp, Artificial sequence
  Probe sequence
cactctcgactgccggcgacggccgggtatgggcccgacgctccagcgcc

SEQ ID NO: 103 - DNA, 50 bp, Artificial sequence
  Probe sequence
atccattttcagggctagttgattcggcaggtgagttgttacacactcct

SEQ ID NO: 104 - DNA, 50 bp, Artificial sequence
  Probe sequence
tagcggattccgacttccatggccaccgtcctgctgtctatatcaaccaa

SEQ ID NO: 105 - DNA, 50 bp, Artificial sequence
  Probe sequence
caccttttctggggtctgatgagcgtcggcatcgggcgccttaacccggc

SEQ ID NO: 106 - DNA, 50 bp, Artificial sequence
  Probe sequence
gttcggttcatcccgcagcgccagttctgcttaccaaaagtggcccacta

SEQ ID NO: 107 - DNA, 50 bp, Artificial sequence
  Probe sequence
ggcactcgcattccacgcccggctccacgccagcgagccgggcttcttac

SEQ ID NO: 108 - DNA, 50 bp, Artificial sequence
  Probe sequence
ccatttaaagtttgagaataggttgagatcgtttcggccccaagacctct

SEQ ID NO: 109 - DNA, 50 bp, Artificial sequence
  Probe sequence
aatcattcgctttaccggataaaactgcgtggcgggggtgcgtcgggtct

SEQ ID NO: 110 - DNA, 50 bp, Artificial sequence
  Probe sequence
gcgagagcgccagctatcctgagggaaacttcggagggaaccagctacta

SEQ ID NO: 111 - DNA, 50 bp, Artificial sequence
  Probe sequence
gatggttcgattagtctttcgcccctatacccaggtcggacgaccgattt

SEQ ID NO: 112 - DNA, 50 bp, Artificial sequence
  Probe sequence
gcacgtcaggaccgctacggacctccaccagagtttcctctggcttcgcc

SEQ ID NO: 113 - DNA, 50 bp, Artificial sequence
  Probe sequence
ctgcccaggcatagttcaccatctttcgggtcctaacacgtgcgctcgtg

SEQ ID NO: 114 - DNA, 50 bp, Artificial sequence
  Probe sequence
ctccacctccccggcgcggcgggcgagacgggccggtggtgcgccctcgg

SEQ ID NO: 115 - DNA, 50 bp, Artificial sequence
  Probe sequence
cggactggagaggcctcgggatcccacctcggccggcgagcgcgccggcc

SEQ ID NO: 116 - DNA, 50 bp, Artificial sequence
  Probe sequence
ttcaccttcattgcgccacggcggctttcgtgcgagcccccgactcgcgc

SEQ ID NO: 117 - DNA, 50 bp, Artificial sequence
  Probe sequence
acgtgttagactccttggtccgtgtttcaagacgggtcgggtgggtagcc

SEQ ID NO: 118 - DNA, 49 bp, Artificial sequence
  Probe sequence
gacgtcgccgccgaccccgtgcgctcgctccgccgtccccctcttcggg

SEQ ID NO: 119 - DNA, 50 bp, Artificial sequence
  Probe sequence
gacgcgcgcgtggccccgagagaacctcccccgggcccgacggcgcgacc

SEQ ID NO: 120 - DNA, 50 bp, Artificial sequence
  Probe sequence
cgcccggggcgcactggggacagtccgccccgccccccgacccgcgcgcg

SEQ ID NO: 121 - DNA, 50 bp, Artificial sequence
  Probe sequence
gcaccccccccgtcgccggggcgggggcgcggggaggaggggtgggagag

SEQ ID NO: 122 - DNA, 50 bp, Artificial sequence
  Probe sequence
cggtcgcgccgtgggaggggtggcccggcccccccacgaggagacgccgg

SEQ ID NO: 123 - DNA, 50 bp, Artificial sequence
  Probe sequence
cgcgcccccgcgggggagacccccctcgcgggggattccccgcgggggtg

SEQ ID NO: 124 - DNA, 50 bp, Artificial sequence
  Probe sequence
ggcgccgggaggggggagagcgcggcgacgggtctcgctccctcggcccc

SEQ ID NO: 125 - DNA, 50 bp, Artificial sequence
  Probe sequence
gggattcggcgagtgctgctgccgggggggctgtaacactcggggrgggt

SEQ ID NO: 126 - DNA, 50 bp, Artificial sequence
  Probe sequence
ttcggtcccgccgcccccgccgccgccgccaccgccgccgccgccgccgc

SEQ ID NO: 127 - DNA, 50 bp, Artificial sequence
  Probe sequence
cccgacccgcgcgccctcccgagggaggacgcggggccggggggcggaga

SEQ ID NO: 128 - DNA, 50 bp, Artificial sequence
  Probe sequence
cgggggaggaggaggacggacggacggacggggccccccgagccaccttc

SEQ ID NO: 129 - DNA, 50 bp, Artificial sequence
  Probe sequence
cccgccgggccttcccagccgtcccggagccggtcgcggcgcaccgccgc

SEQ ID NO: 130 - DNA, 50 bp, Artificial sequence
  Probe sequence
ggtggaaatgcgcccggcggcggccggtcgccggtcgggggacggtcccc

SEQ ID NO: 131 - DNA, 50 bp, Artificial sequence
  Probe sequence
cgccgaccccacccccggccccgcccgcccacccccgcacccgccggagc

SEQ ID NO: 132 - DNA, 50 bp, Artificial sequence
  Probe sequence
ccgccccctccggggaggaggaggaggggcggcgggggaagggagggcgg

SEQ ID NO: 133 - DNA, 50 bp, Artificial sequence
  Probe sequence
gtggaggggtcgggaggaacggggggcgggaaagatccgccgggccgccg

SEQ ID NO: 134 - DNA, 50 bp, Artificial sequence
  Probe sequence
acacggccggacccgccgccgggttgaatcctccgggcggactgcgcgga

SEQ ID NO: 135 - DNA, 50 bp, Artificial sequence
  Probe sequence
ccccacccgtttacctcttaacggtttcacgccctcttgaactctctctt

SEQ ID NO: 136 - DNA, 50 bp, Artificial sequence
  Probe sequence
caaagttcttttcaactttcccttacggtacttgttgactatcggtctcg

SEQ ID NO: 137 - DNA, 50 bp, Artificial sequence
  Probe sequence
tgccggtatttagccttagatggagtttaccacccgctttgggctgcatt

SEQ ID NO: 138 - DNA, 50 bp, Artificial sequence
  Probe sequence
cccaagcaacccgactccgggaagacccgggcgcgcgccggccgctaccg

SEQ ID NO: 139 - DNA, 50 bp, Artificial sequence
  Probe sequence
gcctcacaccgtccacgggctgggcctcgatcagaaggacttgggccccc

SEQ ID NO: 140 - DNA, 50 bp, Artificial sequence
  Probe sequence
cacgagcggcgccggggagcgggtcttccgtacgccacatgtcccgcgcc

SEQ ID NO: 141 - DNA, 50 bp, Artificial sequence
  Probe sequence
ccgcggggcggggattcggcgctgggctcttccctgttcactcgccgtta

SEQ ID NO: 142 - DNA, 50 bp, Artificial sequence
  Probe sequence
ctgagggaatcctggttagtttcttttcctccgctgactaatatgcttaa

SEQ ID NO: 143 - DNA, 50 bp, Artificial sequence
  Probe sequence
gactaatatgcttaaattcagcgggtcgccacgtctgatctgaggtcgcg

SEQ ID NO: 144 - DNA, 50 bp, Artificial sequence
  Probe sequence
aaaccctgttcttgggtgggtgtgggtataatactaagttgagatgatat

SEQ ID NO: 145 - DNA, 50 bp, Artificial sequence
  Probe sequence
catttacgggggaaggcgctttgtgaagtaggccttatttctcttgtcct

SEQ ID NO: 146 - DNA, 50 bp, Artificial sequence
  Probe sequence
  misc_feature (23)..(23): n is a, c, g, or t
ttcgtacagggaggaatttgaangtagatagaaaccgacctggattactc

SEQ ID NO: 147 - DNA, 50 bp, Artificial sequence
  Probe sequence
cggtctgaactcagatcacgtaggactttaatcgttgaacaaacgaacct

SEQ ID NO: 148 - DNA, 50 bp, Artificial sequence
  Probe sequence
ttaatagcggctgcaccatcgggatgtcctgatccaacatcgaggtcgta

SEQ ID NO: 149 - DNA, 50 bp, Artificial sequence
  Probe sequence
aaccctattgttgatatggactctagaataggattgcgctgttatcccta

SEQ ID NO: 150 - DNA, 50 bp, Artificial sequence
  Probe sequence
gggtaacttgttccgttggtcaagttattggatcaattgagtatagtagt

SEQ ID NO: 151 - DNA, 50 bp, Artificial sequence
  Probe sequence
tcgctttgactggtgaagtcttagcatgtactgctcggaggttgggttct

SEQ ID NO: 152 - DNA, 50 bp, Artificial sequence
  Probe sequence
gctccgaggtcgccccaaccgaaatttttaatgcaggtttggtagtttag

SEQ ID NO: 153 - DNA, 50 bp, Artificial sequence
  Probe sequence
gacctgtgggtttgttaggtactgtttgcattaataaattaaagctccat

SEQ ID NO: 154 - DNA, 50 bp, Artificial sequence
  Probe sequence
agggtcttctcgtcttgctgtgttatgcccgcctcttcacgggcaggtca

SEQ ID NO: 155 - DNA, 50 bp, Artificial sequence
  Probe sequence
atttcactggttaaaagtaagagacagctgaaccctcgtggagccattca

SEQ ID NO: 156 - DNA, 50 bp, Artificial sequence
  Probe sequence
tacaggtccctatttaaggaacaagtgattatgctacctttgcacggtta

SEQ ID NO: 157 - DNA, 50 bp, Artificial sequence
  Probe sequence
gggtaccgcggccgttaaacatgtgtcactgggcaggcggtgcctctaat

SEQ ID NO: 158 - DNA, 50 bp, Artificial sequence
  Probe sequence
actggtgatgctagaggtgatgtttttggtaaacaggcggggtaagattt

SEQ ID NO: 159 - DNA, 50 bp, Artificial sequence
  Probe sequence
gccgagttccttttactttttttaacctttccttatgagcatgcctgtgt

SEQ ID NO: 160 - DNA, 50 bp, Artificial sequence
  Probe sequence
tgggttgacagtgagggtaataatgacttgttggttgattgtagatattg

SEQ ID NO: 161 - DNA, 50 bp, Artificial sequence
  Probe sequence
ggctgttaattgtcagttcagtgttttaatctgacgcaggcttatgcgga

SEQ ID NO: 162 - DNA, 50 bp, Artificial sequence
  Probe sequence
ggagaatgttttcatgttacttatactaacattagttcttctatagggtg

SEQ ID NO: 163 - DNA, 50 bp, Artificial sequence
  Probe sequence
atagattggtccaattgggtgtgaggagttcagttatatgtttgggattt

SEQ ID NO: 164 - DNA, 50 bp, Artificial sequence
  Probe sequence
tttaggtagtgggtgttgagcttgaacgctttcttaattggtggctgctt

SEQ ID NO: 165 - DNA, 50 bp, Artificial sequence
  Probe sequence
ttaggcctactatgggtgttaaattttttactctctctacaaggtttttt

SEQ ID NO: 166 - DNA, 50 bp, Artificial sequence
  Probe sequence
cctagtgtccaaagagctgttcctctttggactaacagttaaatttacaa

SEQ ID NO: 167 - DNA, 49 bp, Artificial sequence
  Probe sequence
gggatttagagggttctgtgggcaaatttaaagttgaactaagattcta

SEQ ID NO: 168 - DNA, 50 bp, Artificial sequence
  Probe sequence
tcttggacaaccagctatcaccaggctcggtaggtttgtcgcctctacct

SEQ ID NO: 169 - DNA, 50 bp, Artificial sequence
  Probe sequence
ataaatcttcccactattttgctacatagacgggtgtgctcttttagctg

SEQ ID NO: 170 - DNA, 50 bp, Artificial sequence
  Probe sequence
ttcttaggtagctcgtctggtttcgggggtcttagctttggctctccttg

SEQ ID NO: 171 - DNA, 50 bp, Artificial sequence
  Probe sequence
caaagttatttctagttaattcattatgcagaaggtataggggttagtcc

SEQ ID NO: 172 - DNA, 50 bp, Artificial sequence
  Probe sequence
ttgctatattatgcttggttataatttttcatctttcccttgcggtacta

SEQ ID NO: 173 - DNA, 50 bp, Artificial sequence
  Probe sequence
tatctattgcgccaggtttcaatttctatcgcctatactttatttgggta

SEQ ID NO: 174 - DNA, 50 bp, Artificial sequence
  Probe sequence
aatggtttggctaaggttgtctggtagtaaggtggagtgggtttggggct

SEQ ID NO: 175 - DNA, 50 bp, Artificial sequence
  Probe sequence
gttcgtccaagtgcactttccagtacacttaccatgttacgacttgtctc

SEQ ID NO: 176 - DNA, 50 bp, Artificial sequence
  Probe sequence
ctctatataaatgcgtaggggttttagttaaatgtcctttgaagtatact

SEQ ID NO: 177 - DNA, 50 bp, Artificial sequence
  Probe sequence
tgaggagggtgacgggcggtgtgtacgcgcttcagggccctgttcaacta

SEQ ID NO: 178 - DNA, 50 bp, Artificial sequence
  Probe sequence
agcactctactcttagtttactgctaaatccaccttcgacccttaagttt

SEQ ID NO: 179 - DNA, 50 bp, Artificial sequence
  Probe sequence
cataagggctatcgtagttttctggggtagaaaatgtagcccatttcttg

SEQ ID NO: 180 - DNA, 50 bp, Artificial sequence
  Probe sequence
ccacctcatgggctacaccttgacctaacgtctttacgtgggtacttgcg

SEQ ID NO: 181 - DNA, 50 bp, Artificial sequence
  Probe sequence
cttactttgtagccttcatcagggtttgctgaagatggcggtatataggc

SEQ ID NO: 182 - DNA, 50 bp, Artificial sequence
  Probe sequence
tgagcaagaggtggtgaggttgatcggggtttatcgattacagaacaggc

SEQ ID NO: 183 - DNA, 50 bp, Artificial sequence
  Probe sequence
tcctctagagggatatgaagcaccgccaggtcctttgagttttaagctgt

SEQ ID NO: 184 - DNA, 50 bp, Artificial sequence
  Probe sequence
ggctcgtagtgttctggcgagcagttttgttgatttaactgttgaggttt

SEQ ID NO: 185 - DNA, 50 bp, Artificial sequence
  Probe sequence
agggctaagcatagtggggtatctaatcccagtttgggtcttagctattg

SEQ ID NO: 186 - DNA, 50 bp, Artificial sequence
  Probe sequence
tgtgttcagatatgttaaagccactttcgtagtctattttgtgtcaactg

SEQ ID NO: 187 - DNA, 50 bp, Artificial sequence
  Probe sequence
gagttttttacaactcaggtgagttttagctttattggggagggggtgat

SEQ ID NO: 188 - DNA, 50 bp, Artificial sequence
  Probe sequence
ctaaaacactctttacgccggcttctattgacttgggttaatcgtgtgac

SEQ ID NO: 189 - DNA, 50 bp, Artificial sequence
  Probe sequence
cgcggtggctggcacgaaattgaccaaccctggggttagtatagcttagt

SEQ ID NO: 190 - DNA, 48 bp, Artificial sequence
  Probe sequence
taaactttcgtttattgctaaaggttaatcactgctgtttcccgtggg

SEQ ID NO: 191 - DNA, 50 bp, Artificial sequence
  Probe sequence
tgtggctaggctaagcgttttgagctgcattgctgcgtgcttgatgcttg

SEQ ID NO: 192 - DNA, 50 bp, Artificial sequence
  Probe sequence
ttccttttgatcgtggtgatttagagggtgaactcactggaacggggatg

SEQ ID NO: 193 - DNA, 50 bp, Artificial sequence
  Probe sequence
cttgcatgtgtaatcttactaagagctaatagaaaggctaggaccaaacc

SEQ ID NO: 194 - DNA, 50 bp, Artificial sequence
  Probe sequence
aaagcctacagcacccggtattcccaggcggtctcccatccaagtactaa

SEQ ID NO: 195 - DNA, 50 bp, Artificial sequence
  Probe sequence
ccaggcccgaccctgcttagcttccgagatcagacgagatcgggcgcgtt

SEQ ID NO: 196 - DNA, 50 bp, Artificial sequence
  Probe sequence
ttccgagatcagacgagatcgggcgcgttcagggtggtatggccgtagac

SEQ ID NO: 197 - DNA, 50 bp, Artificial sequence
  Probe sequence
aagcgacgctcagacaggcgtagccccgggaggaacccggggccgcaagt

SEQ ID NO: 198 - DNA, 50 bp, Artificial sequence
  Probe sequence
gcgttcgaagtgtcgatgatcaatgtgtcctgcaattcacattaattctc

SEQ ID NO: 199 - DNA, 50 bp, Artificial sequence
  Probe sequence
gcagctagctgcgttcttcatcgacgcacgagccgagtgatccaccgcta

SEQ ID NO: 200 - DNA, 49 bp, Artificial sequence
  Probe sequence
gccagtcaaatttagcagtggggggttgtataccaactttagtgacact

SEQ ID NO: 201 - DNA, 49 bp, Artificial sequence
  Probe sequence
tgacactaatgttaataagttctgataacccactaccatcggaccagcc

SEQ ID NO: 202 - DNA, 49 bp, Artificial sequence
  Probe sequence
gactagtcaagtgcagtagtgagaaggggggaaagagtagaacaaggag

SEQ ID NO: 203 - DNA, 49 bp, Artificial sequence
  Probe sequence
gaaagagtagaacaaggagttcgatctgtaactgactgtgaacaatcaa

SEQ ID NO: 204 - DNA, 49 bp, Artificial sequence
  Probe sequence
actgactgtgaacaatcaattgagataactcactaccttcggaccagcc

SEQ ID NO: 205 - DNA, 49 bp, Artificial sequence
  Probe sequence
ggctagtcaagtgaagcagtgggagtggagaaggaacaaagaaatctgt

SEQ ID NO: 206 - DNA, 49 bp, Artificial sequence
  Probe sequence
aactggttgtgatcaattagttgtaaacaccactgcactcggaccagcc

SEQ ID NO: 207 - DNA, 49 bp, Artificial sequence
  Probe sequence
cagcaagctagtcaagcgcggttgtggggggagacaatgttaaatcaac

SEQ ID NO: 208 - DNA, 49 bp, Artificial sequence
  Probe sequence
agacaatgttaaatcaacttaacaataacccacaacactcggaccaact

(19) United States
(12) Patent Application Publication
(10) Pub. No.: US 2022/0033809 A1
(43) Pub. Date: Feb. 3, 2022

(54) METHOD AND KIT FOR CONSTRUCTION OF RNA LIBRARY

(71) Applicant: BGI SHENZHEN, Shenzhen (CN)

(72) Inventors: Xi YANG, Shenzhen (CN); Wenjing WANG, Shenzhen (CN); Yanru XING, Shenzhen (CN); Jinjin XU, Shenzhen (CN); Fang CHEN, Shenzhen (CN); Shida ZHU, Shenzhen (CN)

(21) Appl. No.: 17/274,009

(22) PCT Filed: Sep. 5, 2018
(86) PCT No.: PCT/CN2018/104143

§ 371 Date: Mar. 5, 2021

(51) Int. Cl. C12N 15/10; C12Q 1/6876

(52) U.S. Cl. CPC C12N 15/1072; C12N 15/1096; C12Q 1/6876

(57) ABSTRACT

Provided are an RNA library construction method and a specialized kit thereof. The method for preparing an RNA library includes the following steps: extracting RNA and performing fragmentation; adding a tail to 3′ end; ligating an adaptor to 5′ end and hybridizing with a DNA probe mixture; the DNA probe mixture is composed of several DNA probes that are reverse complementary to an RNA that is expected to be removed; removing RNA from the hybrid and removing DNA; performing reverse transcription and PCR amplification to obtain a library solution. The present invention combines polyA tailing and 5′end ligation. The RNA content in the system can be quickly increased by polyA tailing, thereby avoiding subsequent purification losses.

### TECHNICAL FIELD

The present application relates to a method and a kit for construction of an RNA library.

### BACKGROUND ART

Non-invasive testing, including NIPT (non-invasive prenatal testing), is not only a very classic technology, but also an emerging field. Plasma or serum enzymatic testing and NIPT analyze plasma/serum at levels of protein and DNA respectively so as to predict physical health or pregnancy. However, the application of plasma/serum RNA has just started, mainly because RNA is not unstable and plasma/serum RNA is mostly small fragment and its concentration is very low, and thus there is not a very well strategy for construction of library for such low-quality and low-concentration RNA. The current technologies for construction of RNA library are directed to only long RNA or short RNA, and there is not a suitable technology for construction of a library for simultaneous long RNA and short RNA, so that simultaneous study for both long RNA and short RNA requires construction of libraries for them separately.

RNaseH method is a classic method for construction of a library for long RNA. The basic steps comprise: annealing the DNA oligo that is reverse complementary to rRNA with the sample, removing rRNA with RNaseH, and then removing DNA oligo with DNase; reverse transcription (first strand cDNA synthesis), second strand synthesis; gap filling, addition of dA, adaptor ligation, PCR. There are the following problems: the process is very cumbersome, slow, and difficult to the implement; the initial amount of RNA is required to be high; only long fragments of RNA can be detected.

Small RNA library construction comprises the following basic steps: ligating 3′ Adaptor, then ligating 5′ Adaptor, reverse transcription, PCR. There are the following problems: the operation is difficult, slow, and costly; the initial amount of RNA is required to be high; only short fragments of RNA can be detected; serious Bias are induced during the construction of library.

### CONTENTS OF THE PRESENT INVENTION

The present invention provides an RNA library construction method and a specialized kit thereof.

The present invention provides a first method (Method 1) for preparing an RNA library, comprising the following steps:

extracting RNA, and performing fragmentation;

adding a tail to 3′ end;

ligating an adaptor to 5′ end, and hybridizing with a DNA probe mixture; the DNA probe mixture is composed of several DNA probes that are reverse complementary to an RNA that is expected to be removed;

removing RNA from the hybrid and removing DNA;

performing reverse transcription and PCR amplification to obtain a library solution.

The method 1 preferably comprises the following steps:

(1) extracting RNA and performing fragmentation;

(2) adding a tail to 3′ end;

(3) ligating an adaptor to 5′ end and hybridizing with a DNA probe mixture; the DNA probe mixture is composed of several DNA probes that are reverse complementary to an RNA that is expected to be removed;

(4) removing RNA from the hybrid and removing DNA;

(5) performing reverse transcription and PCR amplification in sequence to obtain a library solution.

The fragmentation is implemented by heat treatment. The purpose of the fragmentation is to obtain an RNA fragment of below 400 nt. When RIN>8, the heat treatment has parameters of: 94° C., 10 minutes. When 5≤RIN≤8, the heat treatment has parameters of: 90° C., 5 minutes. When 3≤RIN≤5, the heat treatment has parameters of: 80° C., 5 minutes. When RIN<3, the heat treatment has parameters of: 65° C., 5 minutes.

The step of adding a tail to 3′ end is implemented by the following manner: modifying an RNA fragment to have a phosphate group at its 5′ end and a hydroxyl group at its 3′ end, and then adding a tail. The step of “modifying an RNA fragment to have a phosphate group at its 5′ end and a hydroxyl group at its 3′ end” is implemented by the following method: treating with T4 PNK.

The step of adding a tail refers to adding a poly(A) tail or poly(U) tail.

The step of removing RNA from the hybrid and removing DNA is implemented by the following method: performing RNaseH digestion and DNase digestion sequentially.

The step of extracting RNA refers to extracting CfRNA or extracting total RNA.

The sample used for extracting RNA includes but is not limited to plasma, tissue or cell.

The present invention also seeks to protect a method for preparing an RNA library (Method 2), comprising the following steps:

extracting RNA;

adding a tail to 3′ end;

ligating an adaptor to 5′ end and hybridizing with a DNA probe mixture; the DNA probe mixture is composed of several DNA probes that are reverse complementary to an RNA that is expected to be removed;

removing RNA from the hybrid and removing DNA;

performing reverse transcription and PCR amplification to obtain a library solution.

The method 2 preferably comprises the following steps:

(1) extracting RNA;

(2) adding a tail to 3′ end;

(3) ligating an adaptor to 5′ end and hybridizing with a DNA probe mixture; the DNA probe mixture is composed of several DNA probes that are reverse complementary to an RNA that is expected to be removed;

(4) removing RNA from the hybrid and removing DNA;

(5) performing reverse transcription and PCR amplification in sequence to obtain a library solution.

The step of adding a tail refers to adding a poly(A) tail or poly(U) tail.

The step of removing RNA from the hybrid and removing DNA is implemented by the following method: performing RNaseH digestion and DNase digestion sequentially.

The step of extracting RNA refers to extracting CfRNA or extracting total RNA.

The sample used for extracting RNA includes but is not limited to plasma, tissue or cell.

The present invention also provides a method for preparing an RNA library (Method 3), comprising the following steps:

extracting RNA and performing fragmentation;

adding a tail to 3′ end;

ligating an adaptor to 5′ end and enriching a target RNA fragment;

performing reverse transcription and PCR amplification to obtain a library solution.

The method 3 preferably comprises the following steps:

(1) extracting RNA and performing fragmentation

(2) add a tail to 3′ end;

(3) ligating an adaptor to 5′ end and enriching a target RNA fragment;

(4) performing reverse transcription and PCR amplification in sequence to obtain a library solution.

The fragmentation is implemented by heat treatment. The purpose of the fragmentation is to obtain an RNA fragment of below 400 nt. When RIN>8, the heat treatment has parameters of: 94° C., 10 minutes. When 5≤RIN≤8, the heat treatment has parameters of: 90° C., 5 minutes. When 3≤RIN≤5, the heat treatment has parameters of: 80° C., 5 minutes. When RIN<3, the heat treatment has parameters of: 65° C., 5 minutes.

The step of adding a tail to 3′ end is implemented by the following manner: modifying an RNA fragment to have a phosphate group at its 5′ end and a hydroxyl group at its 3′ end, and then adding a tail. The step of “modifying an RNA fragment to have a phosphate group at its 5′ end and a hydroxyl group at its 3′ end” is implemented by the following method: treating with T4 PNK.

The step of adding a tail refers to adding a poly(A) tail or poly(U) tail.

The step of enriching a target RNA fragment is implemented by a method that includes but is not limited to: chip capture, multiplex PCR, or degenerate PCR.

The step of extracting RNA refers to extracting CfRNA or extracting total RNA.

The sample used for extracting RNA includes but is not limited to plasma, tissue or cell.

The present invention also seeks to protect a method for preparing an RNA library (Method 4), comprising the following steps:

extracting RNA;

adding a tail to 3′ end;

ligating an adaptor to 5′ end and enriching a target RNA fragment;

performing reverse transcription and PCR amplification to obtain a library solution.

The method 4 preferably comprises the following steps:

(1) extracting RNA;

(2) add a tail to 3′ end;

(3) ligating an adaptor to 5′ end and enriching a target RNA fragment;

(4) performing reverse transcription and PCR amplification in sequence to obtain a library solution.

The step of adding a tail refers to adding a poly(A) tail or poly(U) tail.

The step of enriching a target RNA fragment is implemented by a method that includes but is not limited to: chip capture, multiplex PCR, or degenerate PCR.

The step of extracting RNA refers to extracting CfRNA or extracting total RNA.

The sample used for extracting RNA includes but is not limited to plasma, tissue or cell.

Any one of the aforementioned hybrids is a hybrid of DNA and RNA.

Any one of the aforementioned RNAs is a human RNA.

The RNA that is expected to be removed as described above is rRNA and Y RNA. Any one of the aforementioned DNA probe mixtures is composed of 204 kinds of probes, as shown successively in SEQ ID NO: 5 to SEQ ID NO: 208 of the sequence listing.

The RNA that is expected to be removed as described above is rRNA. Any one of the aforementioned DNA probe mixtures is composed of 195 kinds of probes, as shown successively in SEQ ID NO: 5 to SEQ ID NO: 199 of the sequence listing.

Any one of the aforementioned adaptors is shown in SEQ ID NO: 1 of the sequence listing.

Any one of the aforementioned reverse transcriptions uses a reverse transcription primer as shown in SEQ ID NO: 2 of the sequence listing.

Any one of the aforementioned PCRs uses two primers, one is shown in SEQ ID NO: 3 of the sequence listing, and the other is shown in SEQ ID NO: 4 of the sequence listing.

The present invention also seeks to protect a kit (Kit 1) for preparing an RNA library, which comprises a component A, a component B, a component C, a component D, a component E, and a component F.

The present invention also seeks to protect a kit (Kit 2) for preparing an RNA library, which comprises a component A, a component G, a component D, a component E, and a component F.

The present invention also seeks to protect a kit (Kit 3) for preparing an RNA library, which comprises a component A, a component H, a component B, a component C, a component D, a component E, and a component F;

The present invention also seeks to protect a kit (Kit 4) for preparing an RNA library, which comprises a component A, a component H, a component G, a component D, a component E, and a component F.

The present invention also seeks to protect a kit (Kit 5) for preparing an RNA library, which comprises a component I, a component B, a component C, a component D, a component E and a component F.

The present invention also seeks to protect a kit (Kit 6) for preparing an RNA library, which comprises a component I, a component G, a component D, a component E, and a component F.

Component A is a reagent or a combination of reagents for adding a tail to the 3′ end of an RNA molecule.

Component B is a reagent or a combination of reagents for ligating an adaptor to the 5′ end of an RNA molecule.

Component C is a DNA probe mixture. The DNA probe mixture is composed of several DNA probes that are reverse complementary to an RNA that is expected to be removed.

Component D is a reagent or a combination of reagents for removing RNA from the hybrid and removing DNA.

Component E is a reagent or a combination of reagents for reverse transcription.

Component F is a reagent or a combination of reagents for PCR.

Component G is a reagent or a combination of reagents for ligating an adaptor to the 5′ end of an RNA molecule and for hybridizing with a DNA probe.

Component H is a reagent or a combination of reagents for modifying an RNA fragment to have a phosphate group at its 5′ end and a hydroxyl group at its 3′ end.

Component I is a reagent or a combination of reagents for tailing and modifying the 3′ end of an RNA molecule.

Any one of the aforementioned kits further comprises a component J; the component J is a reagent or a combination of reagents for extracting RNA.

The component A is polyA Polymerase and ATP. The component A is RNase H Buffer, ATP, RNase Inhibitor and polyA Polymerase.

The component B is an adaptor and T4 RNA Ligase 1.

The component D is RNase H and DNase. The component D is RNase H, Turbo DNase and RNase-free DNase I. The component D is RNase H, Turbo DNase, RNase-free DNase I, CaCl₂) and Tris-Cl buffer.

The component E is a reverse transcription primer and HiScript II RT. The component E is a reverse transcription primer, dNTP, RNase Inhibitor, HiScript II Buffer and HiScript II RT.

The component F is an amplification primer pair. The component F is an amplification primer pair and HiFi PCR Mix.

The component G is an adaptor, a DNA probe mixture and T4 RNA Ligase 1. The component G is an adaptor, a DNA probe mixture, T4 RNA Ligase 1, T4 RNA Ligase Buffer, ATP, PEG8000, RNase Inhibitor, alkaline phosphatase and Tris-Cl buffer.

The component H is T4 PNK.

The component I is polyA Polymerase, ATP and T4 PNK. The component I is RNase H

Buffer, ATP, RNase Inhibitor, T4 PNK and polyA Polymerase.

The step of adding a tail refers to adding a poly(A) tail or poly(U) tail.

The step of extracting RNA refers to extracting CfRNA or extracting total RNA.

The sample used for extracting RNA includes but is not limited to plasma, tissue or cell.

Any one of the aforementioned hybrids is a hybrid of DNA and RNA.

Any one of the aforementioned RNAs is a human RNA.

The RNA that is expected to be removed as described above is rRNA and Y RNA. Any one of the aforementioned DNA probe mixtures is composed of 204 kinds of probes, as shown successively in SEQ ID NO: 5 to SEQ ID NO: 208 in the sequence listing.

The RNA that is expected to be removed as described above is rRNA. Any one of the aforementioned DNA probe mixtures is composed of 195 kinds of probes, as shown successively in SEQ ID NO: 5 to SEQ ID NO: 199 in the sequence listing.

Any one of the aforementioned adaptors is shown in SEQ ID NO: 1 of the sequence listing.

Any one of the aforementioned reverse transcription primers is shown in SEQ ID NO: 2 of the sequence listing.

For any one of the aforementioned amplification primer pairs, one is shown in SEQ ID NO: 3 of the sequence listing, and the other is shown in SEQ ID NO: 4 of the sequence listing.

The present invention also seeks to protect a use of any one of the aforementioned methods in sequencing. The sequencing is high-throughput sequencing.

The present invention also seeks to protect a use of any one of the aforementioned kits in sequencing. The sequencing is high-throughput sequencing.

The present invention combines polyA tailing and 5′ end ligation. The RNA content in the system can be quickly increased by polyA tailing, thereby avoiding subsequent purification losses. It is convenient to remove specific sequences. Through fragmentation and modification (T4 PNK treatment), all types of RNA enter a similar chemical state, and can be constructed into library at the same time.

### BRIEF DESCRIPTION OF THE DRAWINGS

FIG. 1 shows a schematic flowchart of the method provided by the present invention.

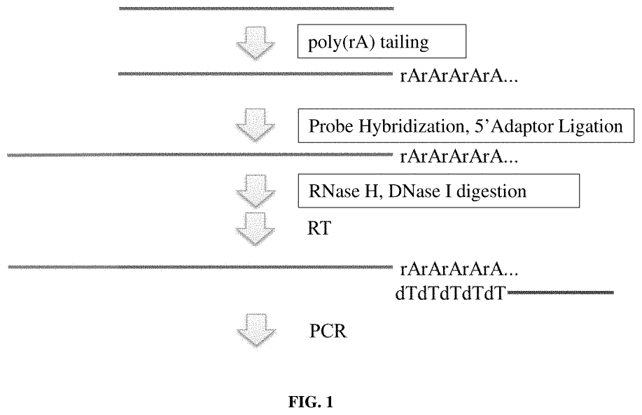

### BEST MODELS FOR CARRYING OUT THE PRESENT INVENTION

The following examples facilitate a better understanding of the present invention, but do not limit the present invention. The experimental methods in the following examples are conventional methods unless otherwise specified. The test materials used in the following examples, unless otherwise specified, are all purchased from conventional biochemical reagent stores. The quantitative tests in the following examples are all set to three repeated experiments, and the results are averaged.

10×RNase H Buffer, RNase H: NEB M0297L. RNase Inhibitor: Thermo EO0382. T4 PNK: NEB M0201. polyA Polymerase: NEB M0276. 10×T4 RNA Ligase Buffer, T4 RNA Ligase 1: NEB M0204. The alkaline phosphatase used in the examples was FastAP alkaline phosphatase: Thermo EF0651. Turbo DNase: Thermo AM2238. RNase-free DNase I: NEB M0303. RNA Clean Beads: Vazyme N411. DNA Clean Beads: Vazyme N412. HiScript II RT, 5×HiScript II Buffer: Vazyme R201. 2×HiFi PCR Mix: KAPA KK2602. Qubit HS: Thermo Q32854.

Small-fragment RNA refers to an RNA molecule with a size of 18 nt to 300 nt (including 18 nt and 300 nt). Common small-fragment RNAs include but are not limited to microRNA, short noncoding RNA (scRNA, tRNA, snRNA, snoRNA, piRNA).

Large-fragment RNA refers to an RNA molecule with a size of larger than 300 nt. Common large-fragment RNAs include but are not limited to mRNA and LncRNA (long noncoding RNA).

In SEQ ID NO: 5 to SEQ ID NO: 208 in the sequence listing, R represents A or G.

### Example 1: Using CfRNA as Sample to Construct RNA Library (Containing Information of Small-Fragment RNA)

I. RNA Extraction

CfRNA was extracted from 300 μL of plasma (the amount of RNA was about 12 ng).

II. RNA Library Construction

1. Adding polyA.

The CfRNA obtained in step I was taken, added with DEPC H₂O to reach a volume of 14.5 μL, then added with 2 μL of 10×RNase H Buffer, then placed on ice, then added with the reagents according to Table 1, and then allowed to react at 37° C. for 30 minutes, then placed on ice, and then added with 2 μL of 40 mM EDTA aqueous solution to stop the reaction.

TABLE 1

| Reagent | Volume |
|---|---|
| 10 mM ATP | 2 μL |
| 40 U/μL RNase Inhibitor | 0.5 μL |
| DEPC water | 0.5 μL |
| 5 U/μL polyA Polymerase | 0.5 μL |

2. Hybridization with rRNA Probe and Ligation of Adaptor.

After the completion of step 1, the system was added with 1 μL of Depletion Probe Set Mix and 0.5 μL of 20 μM adaptor and allowed to react (reaction conditions: 95° C. for 5 minutes, then cooled down to 22° C. at a rate of 0.1° C./sec, and then held at 22° C. for 5 minutes), then placed on ice, then added with the reagents according to Table 2, and allowed to react for 1 hour at 25° C., then added with 2 μL of 1 U/μL alkaline phosphatase and 13 μL of Tris-Cl buffer (pH7.5, 10 mM), and then allowed to react at 37° C. for 10 minutes.

adaptor (SEQ ID NO: 1 in the sequence listing):

5′-GAACGACAUGGCUACGAUCCGACUUNNNN-3′.

In the adaptor, N represents A, G, C or U. Both the 5′ end and the 3′ end of the adaptor have hydroxyl group.

TABLE 2

| Reagent | Volume |
|---|---|
| 10 × T4 RNA Ligase Buffer | 4 μL |
| 10 mM ATP | 4 μL |
| 50% PEG8000 | 7 μL |
| 10 U/μL T4 RNA Ligase 1 | 1 μL |
| 40 U/μL RNase Inhibitor | 0.5 μL |

Depletion Probe Set Mix was a probe mixture solution (the probe mixture was composed of 204 kinds of probes, as shown successively in SEQ ID NO: 5 to SEQ ID NO: 208 of the sequence listing; in the Depletion Probe Set Mix, the concentration of each probe was 0.5 μM). Each probe in the primer mixture was reverse complementary to an RNA (rRNA and Y RNA) that was expected to be removed, and annealed to form a DNA:RNA hybrid.

3. Removal of DNA and RNA Expected to be Removed

(1) After the completion of step 2, the system was added with 1 μL of 5 U/μL RNase H and 4 μL of Tris-Cl buffer (pH7.5, 10 mM), then allowed to react at 37° C. for 30 minutes, then placed on ice, and then added with the reagents according to Table 3, then allowed to react at 37° C. for 30 minutes, then placed on ice.

TABLE 3

| Reagent | Volume |
|---|---|
| 30 mM CaCl₂ | 3 μL |
| 2 U/μL Turbo DNase | 1 μL |
| 2 U/μL RNase-free DNase I | 2 μL |
| Tris-Cl buffer (pH 7.5, 10 mM) | 4 μL |

(2) After the completion of step (1), the system was added with 70 μL of RNA Clean Beads (1×) for purification, the purified product was collected with 15 μL of DEPC water, and 12.5 μL of supernatant was carefully pipetted into a new Nuclease-free centrifuge tube.

4. Reverse Transcription and PCR

(1) The centrifuge tube of step 3 (2) was taken, added with 1 μL of 5 μM Ad153R-25dT-VN, allowed to react at 65° C. for 5 minutes, then placed on ice for 2 minutes, then added with the reagents according to Table 4, and then subjected to reverse transcription (reverse transcription program: 50° C., 40 minutes), then added with 25 μL of 3 mM EDTA aqueous solution, mixed well, and allowed to react at 85° C. for 15 minutes.

TABLE 4

| Reagent | Volume |
|---|---|
| 5 × HiScript II Buffer | 4 μL |
| 10 mM dNTP | 1 μL |
| 40 U/μL RNase Inhibitor | 0.5 μL |
| 200 U/μL HiScript II RT | 1 μL |

Ad153R-25dT-VN (SEQ ID NO: 2 of the sequence listing):

5′-GACCGCTTGGCCTCCGACTTTTTTTTTTTTTTTTTTTTTTTTTV
N-3′.

In the Ad153R-25dT-VN, V represents A, G or C, and N represents A, G, C or T.

(2) After the completion of step (1), 21 μL of product was taken, added with the reagents according to Table 5, and then subjected to PCR amplification (PCR amplification reaction program: 94° C. for 3 min; 94° C. for 20 s, 60° C. for 20 s, 72° C. for 30 s, 20 cycles; 72° C. for 5 min; stored at 16° C.).

TABLE 5

| | Volume |
|---|---|
| 10 μM Pi-Ad153F-FL | 2 μL |
| 10 μM Ad153-RT-Barcode-N | 2 μL |
| 2 × HiFi PCR Mix | 25 μL |

Ad153-RT-Barcode-N(SEQ ID NO: 3 of the sequence listing):

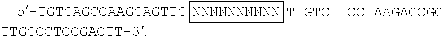

In the Ad153-RT-Barcode-N, the 10 “N”s marked in the bounding box were sample barcode, and each sample had a unique sample barcode.

Pi-Ad153F-FL (SEQ ID NO: 4 of the sequence listing):

5′-GAACGACATGGCTACGATCCGACTT-3′.

(3) After the completion of step (2), the system was added with 90 μL of DNA Clean Beads (1.8×) for purification, and the purified product was collected with 20 μL of 1×TE solution, which was the library solution.

5. Quality Control of Library

Quantitation of the library solution was carried out with Qubit HS, and the library concentration should be greater than 2 ng/μL.

III. High-Throughput Sequencing

The library solutions of multiple samples were mixed and then subjected to high-throughput sequencing.

The instrument used was BGI-SEQ500RS.

The parameters settings were: SE35; 4 pool 1; other settings were BGI-SEQ500RS standard Protocol.

Low-quality Reads were removed; the 4 bases at the 5′ end and the “A”s at the 3′ end (until the first base that was not A) of the Reads were removed; the Reads that contained at least 15 consecutive “A”s were removed; the Reads that were no longer than 17 bases were removed; rRNA alignment and removal were performed;

MicroRNA and piRNA alignments were performed, and the number of Reads of each gene compared to the number of total Reads was calculated.

### Example 2: Comparison of Effects

The plasma samples were 30 human plasma samples.

RNA libraries were constructed according to the method described in Example 1, the average genome alignment rate was 58.72%, and the average transcriptome alignment rate was 39.59%.

Libraries were constructed according to the RNaseH method (http://www.vazyme.com/downloadRepository/11bf871d-c66c-40cc-adbe-317a78af2170.pdf), the average genome alignment rate was 62.44%, and the average transcriptome alignment rate was 26.99% (the alignment rate here was an artifact caused by mitochondrial rRNA, the actual alignment rate was about 3%).

Libraries were constructed according to the Small RNA (http://www.vazyme.com/downloadRepository/32ab9689-8079-4961-b571-daf964c02a46.pdf), the average genome alignment rate was 4.38%, and the average transcriptome alignment rate was 1.77%.

Libraries were constructed according to the SMARTer (SMARTer® Stranded Total RNA-Seq Kit-Pico Input Mammalian), the average genome alignment rate was 35.10%, and the average transcriptome alignment rate was 37.23%.

### Example 3: Using CfRNA as Sample to Construct RNA Library (Containing Both Large-Fragment RNA Information and Small-Fragment RNA Information)

I. RNA Extraction

CfRNA was extracted from 300 μL of plasma (the amount of RNA was about 12 ng).

II. RNA Library Construction

1. Fragmentation, Modification, and Adding polyA.

The fragmentation was implemented by heat treatment.

The purpose of the modification was to make the RNA fragment obtained by the fragmentation form a 5′-end phosphate group and a 3′-end hydroxyl group.

The CfRNA obtained in step I was taken, added with DEPC H₂O to reach a volume of 14.5 μL, then added with 2 μL of 10×RNase HBuffer, subjected to heat treatment (when RIN>8, the heat treatment had parameters of 94° C., 10 minutes; when 5≤RIN≤8, the heat treatment had parameters of 90° C., 5 minutes; when 3≤RIN≤5, the heat treatment had parameters of 80° C., 5 minutes; when RIN<3, the heat treatment had parameters of 65° C., 5 minutes), then placed on ice, and then added with the reagents according to Table 6, then allowed to react at 37° C. for 30 minutes, then placed on ice, and then added with 2 μL of 40 mM EDTA aqueous solution to stop the reaction.

TABLE 6

| Reagent | Volume |
|---|---|
| 10 mM ATP | 2 μL |
| 40 U/μL RNase Inhibitor | 0.5 μL |
| 10 U/μL T4 PNK | 0.5 μL |
| 5 U/μL polyA polymerase | 0.5 μL |

Steps 2 to 5 were the same as steps 2 to 5 of step II in Example 1.

3. High-Throughput Sequencing

Same as the step III of Example 1.

### Example 4. Using Total RNA as Sample to Construct RNA Library

I. RNA Extraction

Total RNA was extracted from a tissue or cells.

II. RNA Library Construction

1. Fragmentation, Modification, and Adding polyA.

The fragmentation was implemented by heat treatment.

The purpose of the modification was to make the RNA fragment obtained by the fragmentation form a 5′-end phosphate group and a 3′-end hydroxyl group.

The total RNA (about 12 ng) obtained in step I was taken, added with DEPC H₂O to reach a volume of 14.5 μL, then added with 2 μL of 10×RNase HBuffer, subjected to heat treatment (when RIN>8, the heat treatment had parameters of 94° C., 10 minutes; when 5≤RIN≤8, the heat treatment had parameters of 90° C., 5 minutes; when 3≤RIN≤5, the heat treatment had parameters of 80° C., 5 minutes; when RIN<3, the heat treatment had parameters of 65° C., 5 minutes), then placed on ice, and then added with the reagents according to Table 6, then allowed to react at 37° C. for 30 minutes, then placed on ice, and then added with 2 μL of 40 mM EDTA aqueous solution to stop the reaction.

2. Hybridization with rRNA Probe and Ligation of Adaptor.

After the completion of step 1, the system was added with 1 μL of Depletion Probe Set Mix and 0.5 μL of 20 μM adaptor and allowed to react (reaction conditions: 95° C. for 5 minutes, then cooled down to 22° C. at a rate of 0.1° C./sec, and then held at 22° C. for 5 minutes), then placed on ice, then added with the reagents according to Table 2, then allowed to react for 1 hour at 25° C., then added with 2 μL of 1 U/μL alkaline phosphatase and 13 μL of Tris-Cl buffer (pH7.5, 10 mM), then allowed to react at 37° C. for 10 minutes.

adaptor (SEQ ID NO: 1 in the sequence listing):

5′-GAACGACAUGGCUACGAUCCGACUUNNNN-3′.

In the adaptor, N represents A, G, C or U. Both the 5′ end and the 3′ end of the adaptor have hydroxyl group.

Depletion Probe Set Mix was a probe mixture solution (the probe mixture was composed of 195 kinds of probes, as shown successively in SEQ ID NO: 5 to SEQ ID NO: 199 of the sequence listing; in the Depletion Probe Set Mix, the concentration of each probe was 0.5 μM). Each probe in the primer mixture was reverse complementary to an RNA (rRNA) that was expected to be removed, and annealed to form a DNA:RNA hybrid.

Steps 3 to 5 were the same as steps 3 to 5 of step II of Example 1.

III. High-Throughput Sequencing

The library solutions of multiple samples were mixed and then subjected to high-throughput sequencing.

The instrument used was BGI-SEQ500RS.

The parameter settings were: SE50; 4 pool 1; other settings were BGI-SEQ500RS standard Protocol.

Low-quality Reads were removed; the 4 bases at the 5′ end and the “A”s at the 3′ end (until the first base that was not A) of the Reads were removed; the Reads that contained at least 15 consecutive “A”s were removed; the Reads that were no longer than 17 bases were removed; rRNA alignment and removal were performed;

MicroRNA and piRNA alignments were performed, and the number of Reads of each gene compared to the number of total Reads was calculated.

RNA-RefSeq alignment was performed, alignments of short noncoding RNA, mRNA and Long Noncoding were performed separately, and RPKM was calculated.

### Example 5: Using Total RNA as Sample to Construct RNA Library

I. RNA Extraction

Total RNA was extracted from a tissue or cells.

II. RNA Library Construction

1. Adding polyA.

The total RNA (about 12 ng) obtained in step I was taken, added with DEPC H₂O to reach a volume of 14.5 μL, then added with 2 μL of 10×RNase H Buffer, then placed on ice, then added with the reagents according to Table 1, and allowed to react at 37° C. for 30 minutes, then placed on ice, then added with 2 μL of 40 mM EDTA aqueous solution to stop the reaction.

2. Hybridization with rRNA Probe and Ligation of Adaptor.

Same as step 2 of step II in Example 4.

Steps 3 to 5 were the same as steps 3 to 5 of step II in Example 1.

III. High-Throughput Sequencing

Same as step III in Example 4.

### Example 6: Comparison of Effects

Samples were placental tissue sample and human immortalized leukocyte (by EB virus method) sample.

RNA libraries were constructed according to the method described in Example 4, the genome alignment rate of the placental tissue sample was 66.32%, the microRNA alignment rate was 0.32%, and the mRNA+lncRNA alignment rate was 27.76%.

RNA libraries were constructed according to the method described in Example 5, the genome alignment rate of the placental tissue sample was 79.18%, the microRNA alignment rate was 21.54%, and the mRNA+lncRNA alignment rate was 9.50%.

RNA libraries were constructed according to the method described in Example 4, the genome alignment rate of the human immortalized leukocyte sample was 73.07%%, the microRNA alignment rate was 0.22%, and the mRNA+lncRNA alignment rate was 28.52%.

RNA libraries were constructed according to the method described in Example 5, the genome alignment rate of the human immortalized leukocyte sample was 86.85%, the microRNA alignment rate was 2.41%, and the mRNA+lncRNA alignment rate was 20.40%.

### INDUSTRIAL APPLICATION

The present invention has the following advantages: the library construction has high efficiency and high sensitivity; the process is simplified and greatly accelerated; it is very low-cost, with one reaction material less than 30 yuan; the operation is simple; it is easy to customize, and modules can be adjusted to suit different needs; it has high quality of sequencing; it is capable of detecting long-fragment and short-fragment RNA at the same time.

1. A method for preparing an RNA library, comprising the following steps:
(i) (a) extracting RNA and performing fragmentation; or (b) extracting RNA;
(ii) adding a tail to 3′ end;
(iii) ligating an adaptor to 5′ end, and hybridizing with a DNA probe mixture; the DNA probe mixture is composed of several DNA probes that are reverse complementary to an RNA that is expected to be removed;
(iv) removing RNA from the hybrid and removing DNA;
(v) performing reverse transcription and PCR amplification to obtain a library solution.

2. The method according to claim 1, wherein the fragmentation is implemented by heat treatment.

3. The method according to claim 1, wherein the step of adding a tail to 3′ end is implemented by the following manner: modifying an RNA fragment to have a phosphate group at its 5′ end and a hydroxyl group at its 3′ end, and then adding a tail.

4. The method according to claim 3, wherein: the step of “modifying an RNA fragment to have a phosphate group at its 5′ end and a hydroxyl group at its 3′ end” is implemented by the following method: treating with T4 PNK.

5. The method according to claim 1, wherein the step of removing RNA from the hybrid and removing DNA is implemented by the following method: performing RNaseH digestion and DNase digestion sequentially.

6-7. (canceled)

8. A method for preparing an RNA library, comprising the following steps:
(i) (a) extracting RNA and performing fragmentation; or (b) extracting RNA;
(ii) adding a tail to 3′ end;
(iii) ligating an adaptor to 5′ end and enriching a target RNA fragment;
(iv) performing reverse transcription and PCR amplification to obtain a library solution.

9. (canceled)

10. A kit for preparing an RNA library, comprising
(1) a component A, a component B, a component C, a component D, a component E, and a component F;
(2) a component A, a component G, a component D, a component E, and a component F;
(3) a component A, a component H, a component B, a component C, a component D, a component E, and a component F;
(4) a component A, a component H, a component G, a component D, a component E, and a component F;
(5) a component I, a component B, a component C, a component D, a component E, and a component F;
or,
(6) a component I, a component G, a component D, a component E, and a component F;
wherein:
Component A is a reagent or a combination of reagents for adding a tail to the 3′ end of an RNA molecule;
Component B is a reagent or a combination of reagents for ligating an adaptor to the 5′ end of an RNA molecule;
Component C is a DNA probe mixture; the DNA probe mixture is composed of several DNA probes that are reverse complementary to an RNA that is expected to be removed;
Component D is a reagent or a combination of reagents for removing RNA from the hybrid and removing DNA;
Component E is a reagent or a combination of reagents for reverse transcription;
Component F is a reagent or a combination of reagents for PCR;
Component G is a reagent or a combination of reagents for ligating an adaptor to the 5′ end of an RNA molecule and for hybridizing with a DNA probe;
Component H is a reagent or a combination of reagents for modifying an RNA fragment to have a phosphate group at its 5′ end and a hydroxyl group at its 3′ end;
Component I is a reagent or a combination of reagents for tailing and modifying the 3′ end of an RNA molecule.

11-18. (canceled)

19. The method of claim 1, wherein the method is for sequencing.

20. The method of claim 8, wherein the method is for sequencing.
